(19) United States
(12) Patent Application Publication
(10) Pub. No.: US 2022/0226588 A1
(43) Pub. Date: Jul. 21, 2022

(54) MEDICAL VENTILATOR HAVING IN-SERIES PISTON PUMPS

(71) Applicant: Ample Inc., San Francisco, CA (US)

(72) Inventor: Ankit Srivastava, San Francisco, CA (US)

(21) Appl. No.: 17/648,377

(22) Filed: Jan. 19, 2022

Related U.S. Application Data
(60) Provisional application No. 63/139,025, filed on Jan. 19, 2021.

(51) Int. Cl. A61M 16/00; A61M 16/20; A61M 16/10

(52) U.S. Cl. CPC A61M 16/0072; A61M 16/208; A61M 16/1055; A61M 2205/7509; A61M 2205/502; A61M 2016/0027; A61M 16/0003

(57) ABSTRACT

A ventilator apparatus is disclosed that provides various improvements with regard to features and practicality and overall benefits to patients suffering from respiratory and other conditions. The apparatus is constructed in an efficient and reliable way including a single motor and drivetrain assembly that operates a plurality of fluid pumps to achieve the present ventilator functions as described and shown.

### RELATED APPLICATIONS

This application claims priority to U.S. Provisional Application No. 63/139,025, bearing the present title, filed on Jan. 19, 2021, which is hereby incorporated by reference.

### TECHNICAL FIELD

This application generally relates to medical ventilators.

### BACKGROUND

Existing ventilators are expensive and have long lead times to manufacture. Furthermore, many technical limitations with respect to performance, durability, ease of manufacturing, safety and efficiency exist in current ventilator designs. It is necessary or desirable to overcome these and other deficiencies in the art, especially when the public health requires the scaling up of production of sufficient numbers of suitable ventilators to address pandemics as experienced in recent years.

### SUMMARY

One or more embodiments are directed to a ventilator apparatus, comprising a drive motor acting as a prime mover, receiving energy from a power source and providing a rotational mechanical motor output; a drivetrain, coupled to said drive motor, that receives said rotational mechanical motor output and converts the same into an oscillating linear mechanical movement; an elongated drive shaft, coupled to said drivetrain and driven thereby, the drive shaft further coupled to and powering two fluid pumps including a first (expiratory) fluid pump and a second (inspiratory) fluid pump, said drive shaft disposed in-line with and between said two fluid pumps; wherein said drive shaft translates axially along an axis of the drive shaft according to said oscillating linear mechanical movement of the drivetrain, and wherein said drive shaft forces a linear movement of both of said fluid pumps along said axis; a first fluid pathway that receives an expiratory input volume of fluid into said first (expiratory) fluid pump during a first phase of operation of said apparatus and discharges an expiratory output volume of fluid out of said first (expiratory) fluid pump during a second phase of operation of said apparatus; and a second fluid pathway that receives a breathing gas volume into said second (inspiratory) fluid pump during said first phase of operation of the apparatus and discharges said breathing gas volume during said second phase of operation of the apparatus.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 illustrates an exemplary view of a ventilator system in a first state according to one or more exemplary embodiments.

FIG. 2 illustrates an exemplary view of a ventilator system in a second state according to one or more exemplary embodiments.

FIG. 3 illustrates an exemplary end view of a ventilator system from one end thereof according to one or more embodiments.

FIG. 4 illustrates an exemplary top view of a ventilator system according to one or more embodiments.

FIG. 5 illustrates an exemplary top view of a ventilator system and certain gas pathways and accessories according to one or more embodiments.

FIG. 6 illustrates an exemplary perspective view of the system including its housing as may be used in one or more embodiments.

FIG. 7 describes exemplary settings and corresponding functions accessible using a user interface of the system.

FIG. 8 illustrates another exemplary perspective view of the system and the use of limit switches therein to control the mechanical linear driving motions.

FIG. 9 illustrates yet another exemplary perspective view of one or more embodiments including placement of some gas pressure switches in the gas lines thereof.

FIG. 10 illustrates an exemplary side view of a ventilator system according to one or more embodiments.

FIG. 11 illustrates another exemplary side view of a ventilator system according to one or more embodiments.

FIG. 12 illustrates an end view of a ventilator system and some of the pressure switches used therein according to one or more embodiments.

FIG. 13 illustrates another exemplary top view of a ventilator system and limit switches according to one or more embodiments.

### DETAILED DESCRIPTION

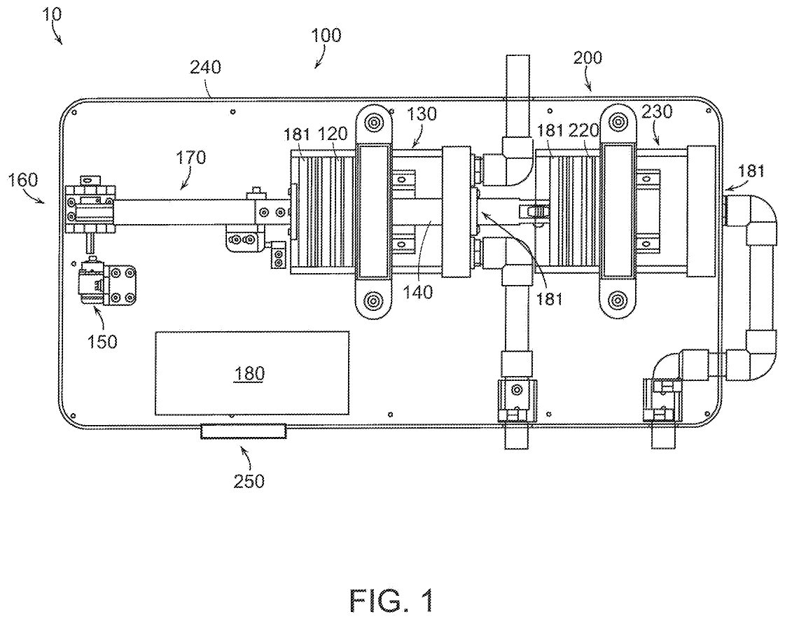

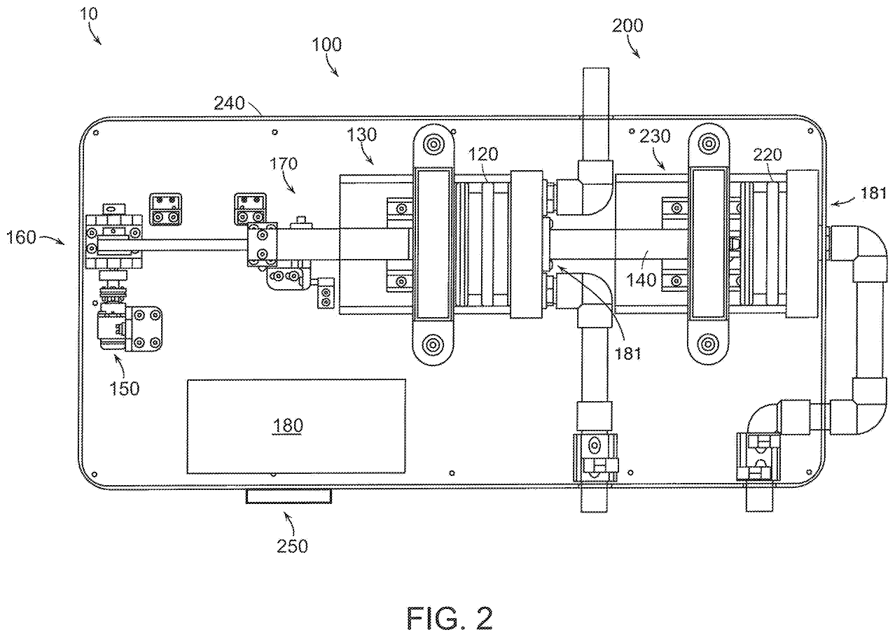

FIG. 1 is a top view of a mechanical ventilator 10 in a first state according to an embodiment. The ventilator 10 includes an expiratory piston pump 100 and an inspiratory piston pump 200. Each pump 100, 200 includes a moveable piston 120, 220 disposed in a cylinder 130, 230, respectively. The cylinders 130, 230 can comprise an acrylic material (e.g., polymethyl methacrylate) or another material. The pistons 120, 220 are mechanically coupled in series with a connecting rod or shaft 140. Since the pistons 120, 220 are mechanically coupled in series, the pistons 120, 220 operate in phase with each other, moving linearly along a main axis of connecting rod or shaft 140. For example, when piston 120 is in an expanded state, piston 220 is also in an expanded state (and vice versa), as illustrated in FIG. 1. Likewise, when piston 120 is in a compressed state, piston 220 is also in a compressed state, as illustrated in FIG. 2. The pistons 120, 220 can comprise stainless steel, plastic, or another material.

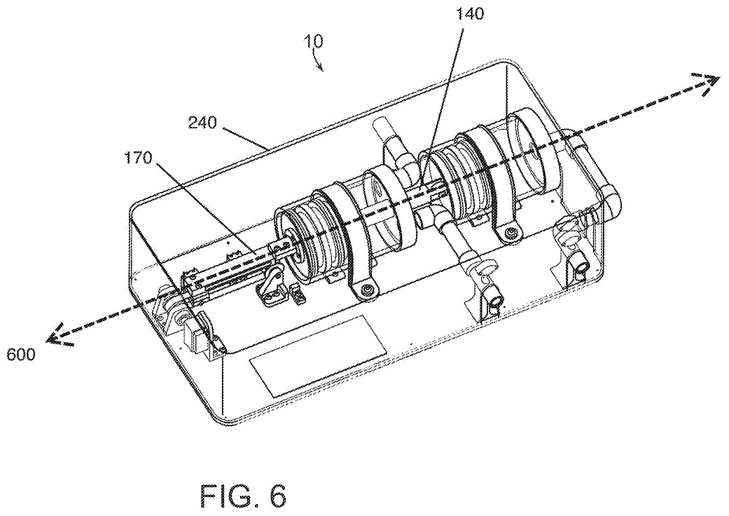

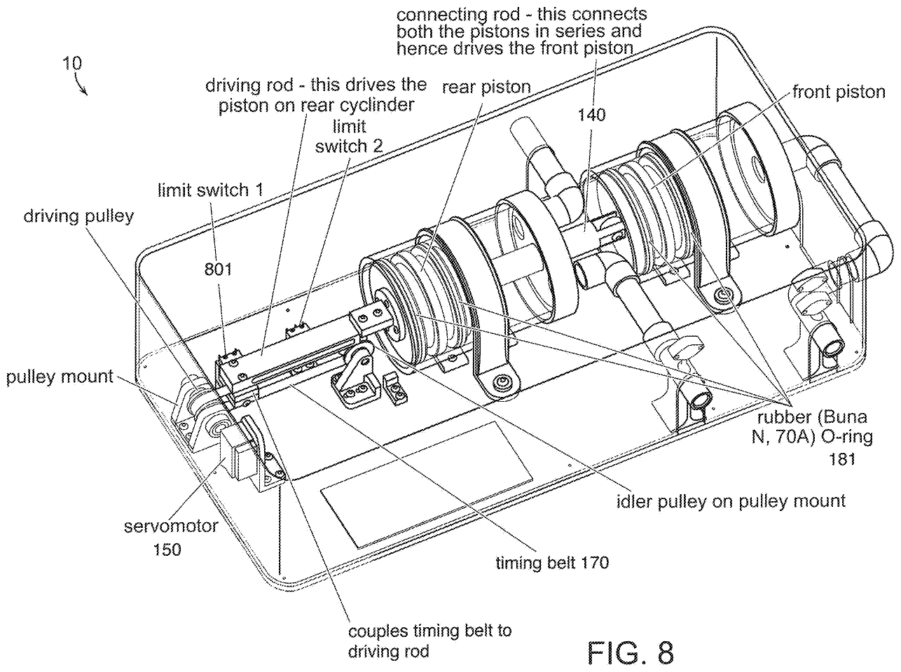

The pistons 120, 220 are driven by a drive motor 150 acting as a prime mover which receives energy from an energy source such as an AC or a DC electric power supply (i.e., utility power outlet and/or battery or similar source). The motor 150 is in mechanical communication with a drive motor assembly 160 acting as a powertrain that takes the rotational motor movement and translates the rotational motion of the motor 150 into a linear motion, in some aspects, oscillating linearly (back and forth) along an axis or direction congruent with a major dimension of the connecting rod or shaft 140. FIG. 6 shows conceptually the direction of axis 600 through or parallel to the main or major axis of the connecting rod shaft, which will also coincide in this exemplary embodiment with the direction of movement of timing belt 170. In an example, the drive motor assembly 160 is in mechanical communication with piston 120 via a timing belt 170. When the motor 150 rotates in a first direction, the linear motion created by the drive motor assembly 160 causes a driving rod (FIG. 8) to push piston 120, and thus piston 220, from the expanded state (or expanded position) to the compressed state (or compressed position). When the motor 150 rotates in a second direction (opposite to the first direction), the linear motion created by the drive motor assembly 160 causes the a driving rod to pull or retract piston 120, and thus piston 220, from the compressed state to the expanded state. The direction and speed of the motor 150 is controlled by a microprocessor-based controller 180 that is in electrical communication with the motor 150 and which in some examples may control a driving voltage, frequency and/or current supplied to the motor 150. Limit switches 1, 2 (FIG. 8) can be used to limit the linear motion of the drive motor assembly 160, for example to limit the position of the driving rod (e.g., a sensor or object on driving rod) relative to the limit switches 1, 2. Limit switch 1 can correspond to the start position of the driving rod (e.g., when the pistons 120, 220 are in the expanded state). Limit switch 2 can correspond to the stop position of the driving rod (e.g., when the pistons 120, 220 are in the compressed state). Additional details regarding the operation of the drive motor assembly 160 are illustrated in FIG. 8. We also note the exemplary embodiment of FIG. 8 which shows the use of limit switches 801 to sense and limit the linear motion of the drivetrain, pistons, and optionally to cause reversal of said movement to switch directions so that the apparatus has the described cyclic movement.

Piston 220 moves in phase with piston 120, as discussed above, since they are mechanically coupled in series. Therefore, pushing piston 120 from a (first) expanded state to a (second) compressed state causes piston 220 to be pushed from a complementary (first) expanded state to a complementary (second) compressed state. Likewise, pulling or retracting piston 120 from the compressed state to the expanded state causes piston 220 to be pulled/retracted from the compressed state to the expanded state.

A plurality of O-rings 181 is used to form fluid-tight seals in each pump 100, 200. For example, O-rings 181 are disposed on each piston 120, 220 to form a fluid-tight seal around each piston 120, 220. The seal may be resistant to unwanted gas flow by said seal as the seal is seated in a dimensionally-matching aperture and maintains a sufficient pressure on any gap between the seal and the aperture or between the seal and the inner shaft to prevent gas flow between opposing sides of said seal. In addition, one or more O-rings 181 is disposed between the connecting rod 140 and cylinder 130 to form a fluid-tight environment within and between the gas volumes in said cylinders. Additional O-rings 181 can be used to seal the fluid connections into and out of each cylinder 130, 230 as shown.

The ventilator system 10 also includes a common housing 240 that houses the components of the system and a user interface 250 disposed on or in the housing 240. The housing 240 is shown open so that the inner components can be seen in the figure, but the housing can comprise a shell or a multi-part base portion onto which the components are secured and an upper portion or lid that fits over the components to close them off within the housing and to prevent damage or contamination to the components. In some embodiments, all of the housing, or alternatively just the upper lid of the housing may be constructed of a transparent material so the workings of the inner parts can be visible during operation. The base and upper parts of the housing may be glued with epoxy to one another, fused using plastic welding methods, or secured to one another with mechanical fasteners such as screws, optionally with a fluid-resistant gasket sealing leakage of fluids into or out of the housing 240.

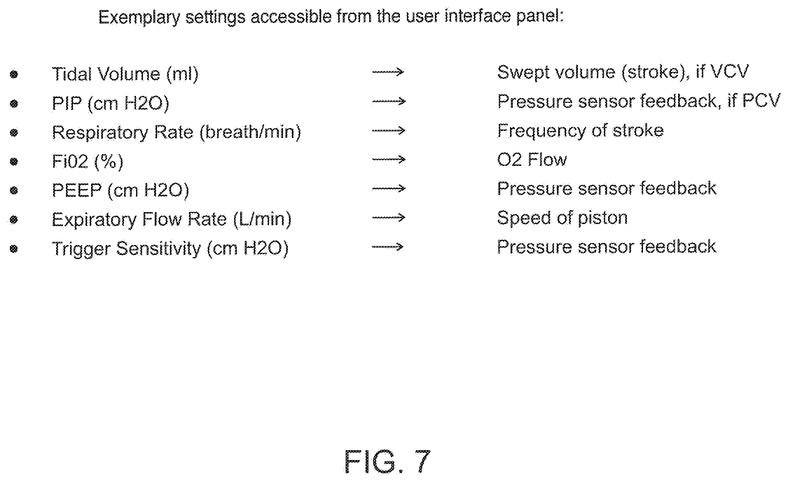

A user interface 250 is electrically coupled to the processor based controller 180 which may be constructed on a printed circuit board (PCB) or other electronic integrated circuit to receive one or more input signals from the user interface 250 to set one or more parameters, settings, and/or operating modes (collectively, settings) of the ventilator. A ribbon connector or printed circuit lines can connect the user interface panel 250 with the internal processor circuits and other electrical components on electrical controller 180. Examples the settings that can be set with the user interface 250 include (1) ventilation operating mode (e.g., volume control with PEEP, pressure control with PEEP, pressure support, and/or another ventilation operating mode), (2) the patient's tidal volume, (3) the positive inhalation pressure set point (e.g., when operating in pressure-control mode), (4) the purified oxygen concentration and flow rate, (5) oxygen concentration in patient's inhalation gas, (6) expiratory flow rate, (7) respiratory rate, (8), I:E ratio, and/or (9) breath pause length. Examples of these and/or other settings are illustrated in FIG. 7 which can be set or modified using the user interface panel and processor based controller. Additional or fewer settings can be provided in other embodiments.

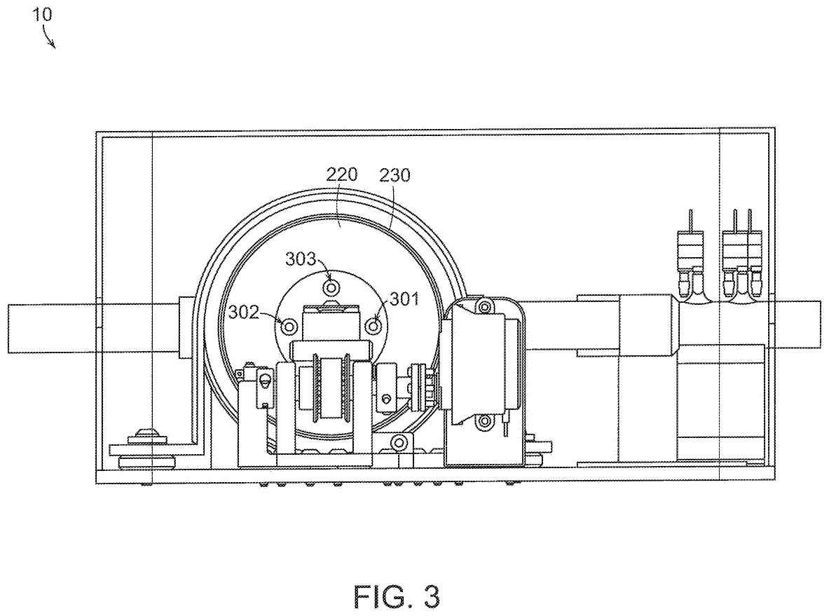

FIG. 3 is a rear side view of the ventilator 10 according to an embodiment. Some components of the overall device described may be removed from certain views for ease of viewing, but the present examples are illustrative of the invention so as to explain to those of skill in the art how these non-limiting embodiments are configured and arranged. Of course, similar or equivalent configurations and arrangements can be equally contemplated, for example by positioning or sizing some of the components differently as suits an application of interest. As illustrated, cylinder 230 includes one-way, e.g., check valves 301-303. Valve 301 can be used to allow ambient or compressed air into cylinder 230. Valve 302 can be used to allow purified oxygen from an oxygen tank or hospital supply line into cylinder 230. Valve 303 can be used to output an oxygen-enriched gas mixture (i.e., a mixture of ambient/compressed air from valve 301 and oxygen from valve 302) to the patient via an inhalation line 310. One-way valves 301 and 302 only allow fluid to flow into cylinder 230. One-way valve 303 only allows fluid to flow out of cylinder 230.

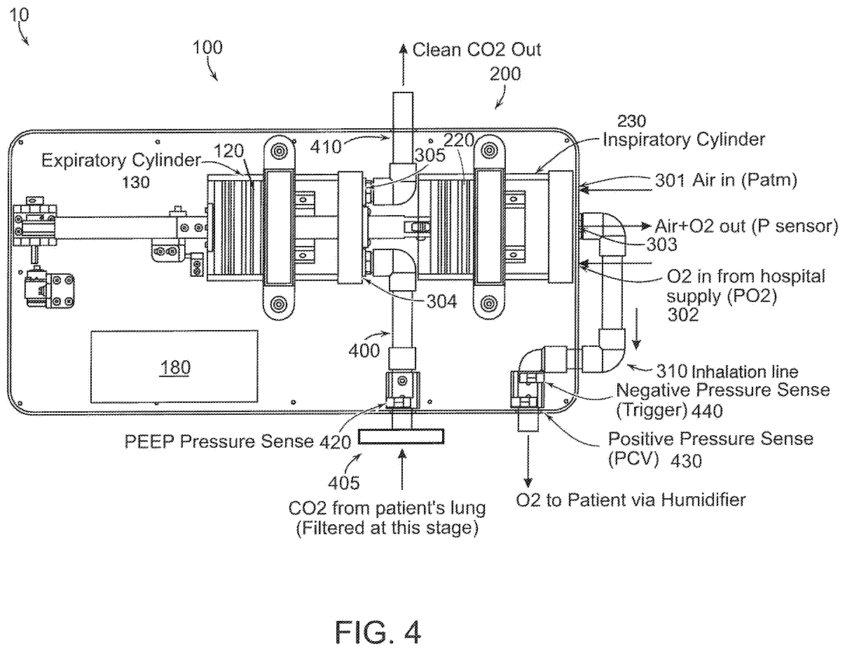

FIG. 4 is a top view of the ventilator 10 to further illustrate the operation of each pump 100, 200 and associated gas flows. When piston 120 transitions from the compressed state to the expanded state, a negative pressure is formed in cylinder 130 to receive the exhaled gas via exhalation line 400. Fluid communication between cylinder 130 and exhalation line or pathway 400 is controlled by a one-way valve 304 that only allows fluid to flow into the cylinder 130. The exhaled gas passes through a replaceable filter 405 (e.g., a bacterial filter) that can filter out droplets, bio materials, contaminants and aerosol particles, such as from patients having a contagious illness (e.g., COVID-19 or another contagious illness). The replaceable filter 405 can be replaced after patient use to reduce the likelihood of cross-contamination.

When piston 220 transitions from the compressed state to the expanded state, a negative pressure is formed in cylinder 230 to receive air and purified oxygen from one-way or check valves 301 and 302, respectively. Thus, cylinders 130, 230 respectively store exhaled gas and oxygen-enriched gas-to-be-inhaled in the next breath concurrently when the respective pistons 120, 220 transition from the compressed state to the expanded state. The valves 301, 302, and 304 close when pistons 120, 220 reach the respective positions corresponding to the expanded state.

When expiratory piston 120 transitions from the expanded state to the compressed state, a positive pressure is formed in cylinder 130 to force the exhaled gas out of cylinder 130 (e.g., into the atmosphere) via output line 410. Fluid communication between cylinder 130 and output line 410 is controlled by a one-way valve 305 that only allows fluid to flow out of the cylinder 130. When piston 220 transitions from the expanded state to the compressed state, a positive pressure is formed in cylinder 230 to force the oxygen-enriched gas-to-be-inhaled into the inhalation line 310 for patient inhalation. Thus, cylinders 130, 230 respectively discharge exhaled gas and oxygen-enriched gas-to-be-inhaled in the next breath concurrently when the respective pistons 120, 220 transition from the expanded state to the compressed state.

The inhalation line 310 can be fluidly coupled to a humidifier to increase the water-vapor content of the oxygen-enriched gas-to-be-inhaled. In some embodiments, a humidifier can be integrated into the ventilator system 10.

In volume-control mode, the processor-based controller 180 determines the position of the pistons 120, 220 to transition from the expanded state to the compressed state based on the patient's tidal volume, which is received by the controller 180 as a user input via user interface 250. The controller 180 can have a user input or can be pre-programmed with the diameters of the cylinders 130, 230 which the controller 180 can use to determine the position of the pistons 120, 220 to form the set-point tidal volume in each cylinder 130, 230 (e.g., the displacement of each piston 120, 220 equals the set-point tidal volume). In one example, each cylinder 130, 230 has approximately a 4-inch diameter. Other diameters of cylinders 130, 230 can also be provided. The position of the pistons 120, 220 can be determined by the number of rotations of motor 150, which can be stored in the memory of controller 180 as a look-up table, a formula, or other relationship. Fully compressing piston 220 therefore results in the delivery of the tidal volume set point to the patient (e.g., via inhalation line 310).

The frequency that the controller 180 transitions the pistons 120, 220 between the compressed state and the expanded state corresponds to the respiratory rate, which is an input setting in user interface 250. Additional input settings that can be used by the controller 180 include the inspiratory-rate-to-expiratory-rate ratio (or I:E ratio) and any pause between inspiration (inhalation) and expiration (exhalation). The controller can determine the expiratory flow rate using the inputs of respiratory rate, I:E ratio, and optionally the breath pause length. The expiratory flow rate corresponds to the speed that the pistons 120, 220 transition (e.g., retract) from the expanded state to the compressed state, which the controller 180 can determine based on the diameter of the cylinders 130, 230. The speed of the pistons 120, 220 can be controlled by adjusting the rotational speed of motor 150, which can be stored in the memory of controller 180 as a look-up table, a formula, or other relationship.

To achieve the desired oxygen concentration in the patient's inhalation gas, the controller 180 can calculate the required purified oxygen flow rate based on the calculated piston 120, 220 retraction speed, the diameter of the cylinders 130, 230, and the purified oxygen concentration. The required purified oxygen flow rate can be displayed on the user interface 250 with instructions for a nurse or other health care professional to set accordingly (e.g., by adjusting a valve in the hospital oxygen line). Alternatively, the valve 302 can be adjusted by the controller 180 (e.g., based on a pressure sensor in the purified oxygen intake line) to achieve the required purified oxygen flow rate.

Each one-way valve 301-305 can be a check valve, a solenoid valve, or another one-way valve. When the one-way valves 301-305 are check valves, the one-way valves 301-305 open and close automatically in response to the relative pressure differential across the respective valve. When the one-way valves 301-305 are solenoid valves, the one-way valves 301-305 open and close in response to electrical control signals sent from the controller 180. Each one-way valve 301-305 has a normally-closed position and an open position. The default for each valve 301-305 is the normally closed-position, and each valve 301-305 opens only in response to a minimum pressure differential across the valve (e.g., in a check valve) or in response to a control signal (e.g., in a solenoid valve).

The ventilator 10 includes pressure sensors that are in electrical communication with the controller 180. For example, a PEEP pressure sensor 420 is located in, or in fluid communication with, the exhalation line 400 to sense the pressure in the exhalation line 400. The controller 180 controls the expansion of piston 120 so that a minimum positive end-expiratory pressure (PEEP) remains in the patient's lungs at the end of the exhalation cycle. The PEEP can be set via the user interface 250 (e.g., a graphical user interface or other interface) on the ventilator 10. Examples of PEEP set points include the range of 3 cm H₂O to 5 cm H₂O, but higher or lower PEEP set points can be used. In operation, the controller 180 stops the rotation of motor 150 to stop pistons 120, 220 from further transitioning to the expanded state (e.g., to the left in FIG. 4) when the feedback from the PEEP pressure sensor 420 indicates that the PEEP pressure in the exhalation line is at the set point PEEP (e.g., 4 cm H₂O) and/or within a tolerance range thereof (e.g., plus or minus 10% of the PEEP set point).

A positive pressure sensor 430 and a negative pressure sensor 440 are located in, or in fluid communication with, the inhalation line 310 to sense the positive and negative pressure, respectively, in the inhalation line 310. The controller 180 controls the compression of piston 220 so that positive inhalation pressure in the inhalation line 310, measured by positive pressure sensor 430, is less than or equal to a positive inhalation pressure set point. The positive inhalation pressure set point can be set via the user interface 250 (e.g., a graphical user interface or other interface) on the ventilator 10. In operation, when the ventilator 10 operates in pressure-control mode, the controller 180 stops the rotation of motor 150 to stop pistons 120, 220 from further transitioning to the compressed state (e.g., to the right in FIG. 4) when the feedback from the positive pressure sensor 430 indicates that the positive pressure in the inhalation line is at the positive inhalation pressure set point (e.g., 20 cm H₂O) and/or within a tolerance range thereof (e.g., plus or minus 10% of the positive inhalation pressure set point). Reaching the positive inhalation pressure set point indicates to the controller 180 that the patient has received or inhaled a predetermined amount of oxygen-enriched gas mixture when the ventilator 10 operates in pressure-control mode.

When the ventilator 10 operates in volume-control mode, the controller 180 uses the positive pressure sensor 430 to generate an alarm when the positive inhalation pressure reaches or exceeds a maximum or peak positive inhalation pressure. The controller 180 does not use the positive inhalation pressure set point in volume-control mode. Otherwise, in volume-control mode, the piston 220 is fully compressed to deliver the entire volume of the oxygen-enriched gas mixture to the patient.

When the ventilator 10 operates in pressure-support mode (e.g., when the patient can initiate a breath, such as when the patient is weaning off ventilator-assisted respiration), the controller 180 uses the negative inhalation pressure sensed by negative pressure sensor 440 as a trigger to determine when the patient has initiated a breath. The trigger causes the controller 180 to begin the inspiration or inhalation cycle by starting the rotation of motor 150 to transition the pistons 120, 220 from the expanded state to the compressed state. The controller 180 does not use the negative inhalation pressure in pressure-control mode or volume-control mode.

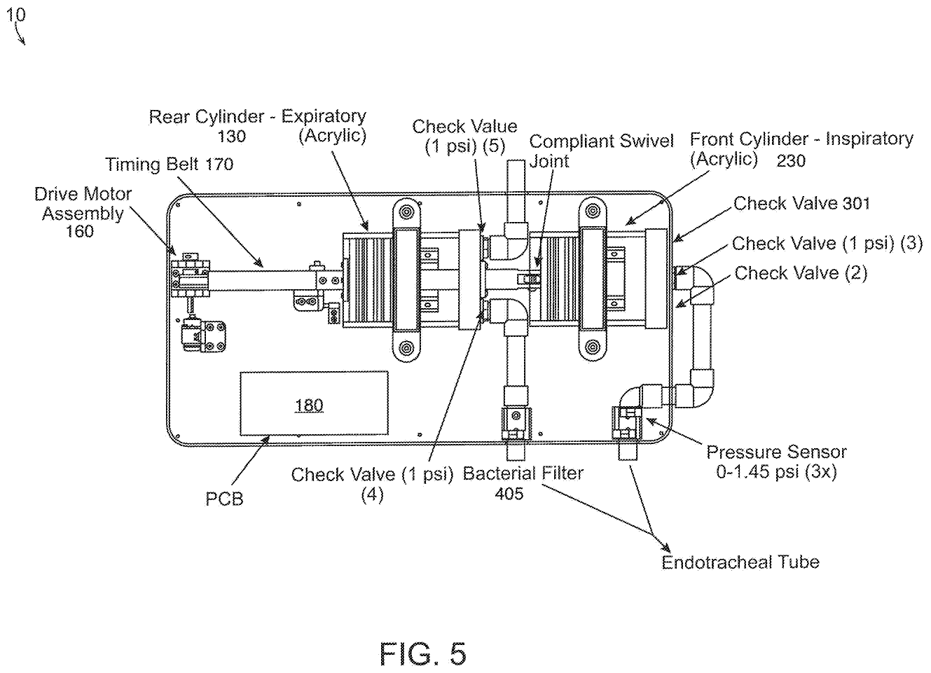

FIG. 5 is another top view of ventilator 10 with example labels and descriptions of certain components thereof including some non-limiting arrangement of gas pathways for clarity.

FIG. 6 illustrates a ventilator system 10 and its placement in housing 240 according to an exemplary arrangement. It should be understood that the mechanical arrangement of the components shown in this and other exemplary embodiments is not limiting, and those skilled in the art can re-arrange or substitute various parts of the invention as described and illustrated without loss of generality.

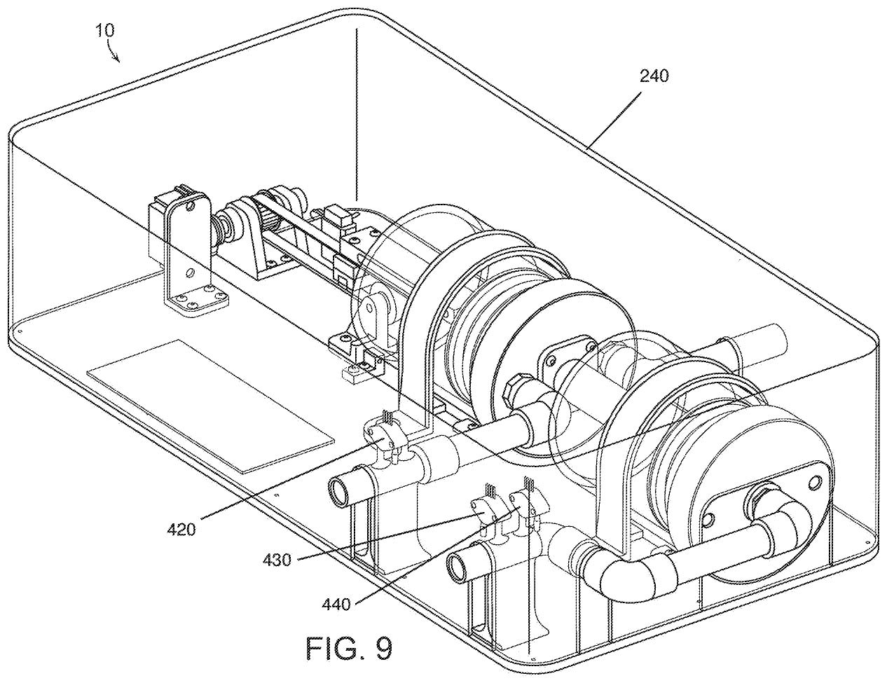

FIG. 9 illustrates another view of system 10 according to an exemplary arrangement. Specifically, we note the use of pressor sensors such as PEEP pressure sensor 420, positive pressure sensor (PCV) 430 and negative pressure sensor 440 which can function as a trigger. Each pressure sensor can sense a gas pressure in a line in which it is placed and provide a corresponding pressure value signal that can be an electrical signal indicating said pressure and can be sent as necessary over a wired or wireless communication line to a display or to processor 180 for triggering a function or for inclusion in a logic operation.

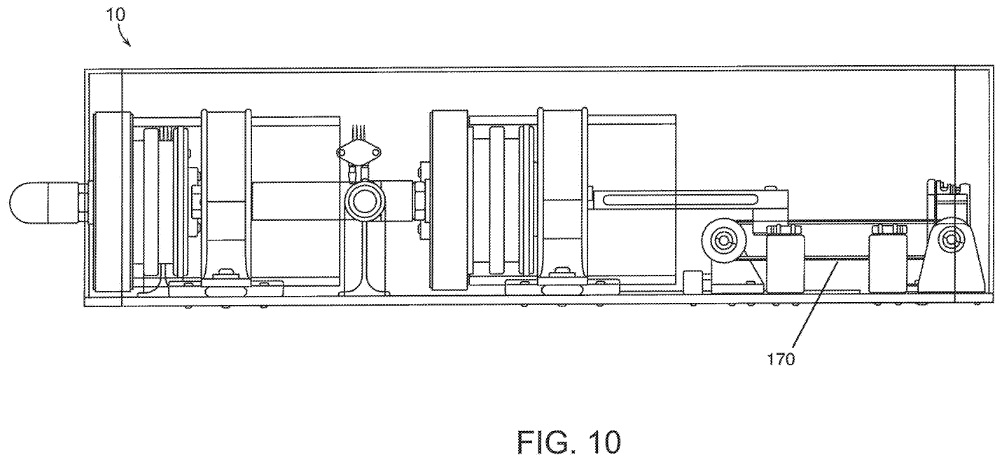

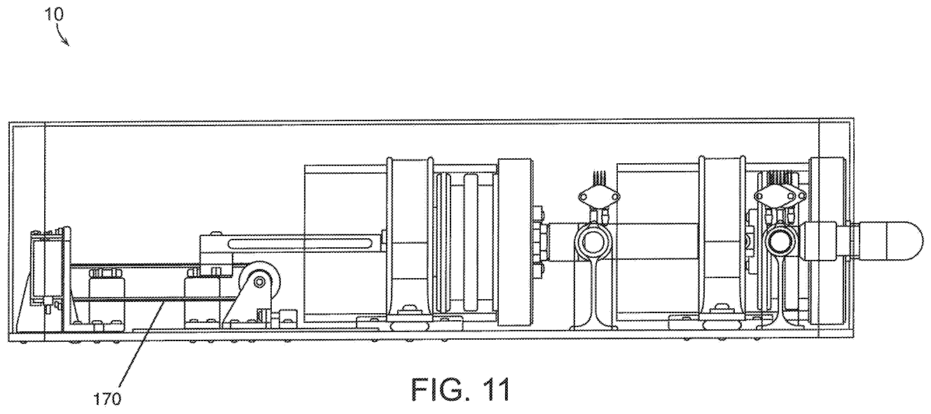

FIGS. 10 and 11 are side views of ventilator 10 taken from opposing ends of the system. We note the side view of timing belt 170 in a preferred embodiment.

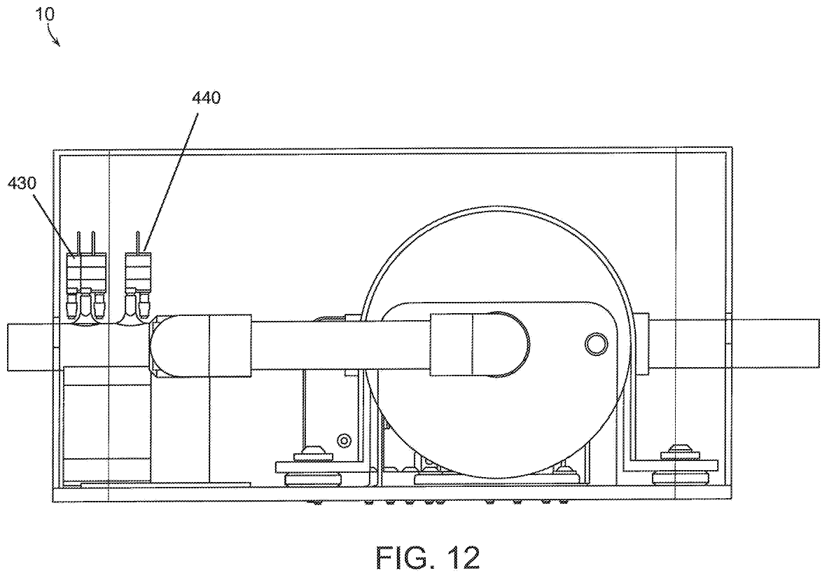

FIG. 12 is a front side view of ventilator from the opposing side as the rear side view illustrated in FIG. 3. We can see the placement of pressure sensors 430 and 440 in an exemplary configuration.

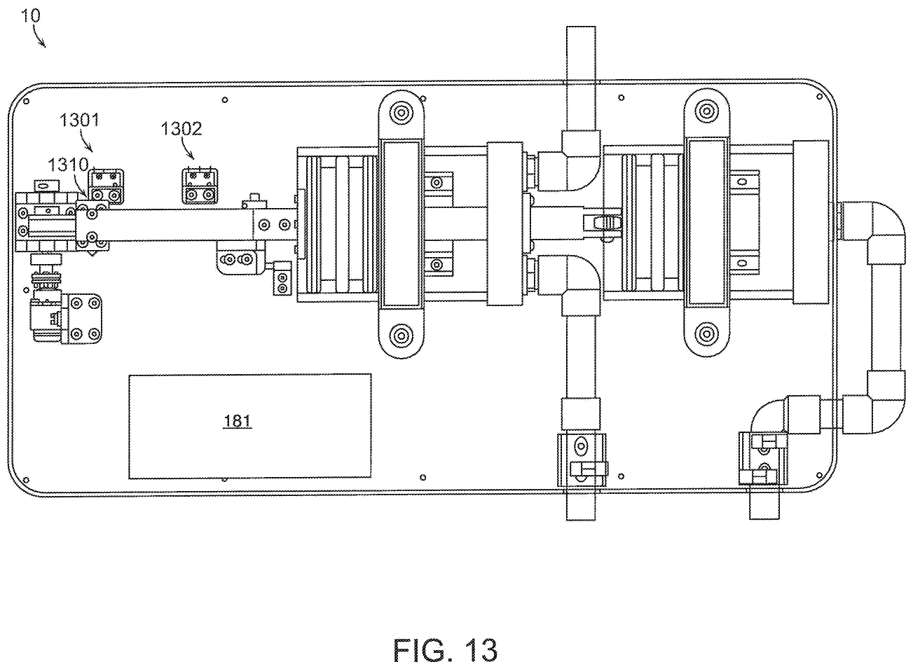

FIG. 13 is a top view of ventilator 10. FIG. 13 is identical to FIG. 1 except that FIG. 13 illustrates the first and second limit switches 1301, 1302 which can limit the backward and forward positions, respectively, of the driving rod (e.g., using position bracket 1310).

As can be seen, a technical advantage of the disclosed ventilator is that it can be manufactured quickly and inexpensively without sacrificing functionality. In addition, the disclosed ventilator is re-usable even with patients that may have infectious diseases such as COVID-19 or other respiratory ailments.

This disclosure should not be considered limited to the particular embodiments described above. Various modifications, equivalent processes, as well as numerous structures to which the technology may be applicable, will be apparent to those skilled in the art to which the technology is directed upon review of this disclosure.

What is claimed is:

1. A ventilator apparatus, comprising:
a drive motor acting as a prime mover, receiving energy from a power source and providing a rotational mechanical motor output;
a drivetrain, coupled to said drive motor, that receives said rotational mechanical motor output and converts the same into an oscillating linear mechanical movement;
an elongated drive shaft, coupled to said drivetrain and driven thereby, the drive shaft further coupled to and powering two fluid pumps including a first (expiratory) fluid pump and a second (inspiratory) fluid pump, said drive shaft disposed in-line with and between said two fluid pumps;
wherein said drive shaft translates axially along an axis of the drive shaft according to said oscillating linear mechanical movement of the drivetrain, and wherein said drive shaft forces a linear movement of both of said fluid pumps along said axis;
a first fluid pathway that receives an expiratory input volume of fluid into said first (expiratory) fluid pump during a first phase of operation of said apparatus and discharges an expiratory output volume of fluid out of said first (expiratory) fluid pump during a second phase of operation of said apparatus; and
a second fluid pathway that receives a breathing gas volume into said second (inspiratory) fluid pump during said first phase of operation of the apparatus and discharges said breathing gas volume during said second phase of operation of the apparatus.

2. The apparatus of claim 1, further comprising a microcontroller circuit configured and arranged to generate an electrical control signal to control the operation of the drive motor.

3. The apparatus of claim 1, further comprising a common housing that encloses the parts of said apparatus.

4. The apparatus of claim 1, further comprising a timing belt wrapped about two rotating wheels wherein one of said rotating wheels is driven by said drive motor.

5. The apparatus of claim 1, further comprising a user interface panel electronically coupled to said microcontroller circuit and which is configured and arranged to accept an input so as to cause a controlled operation of said drive motor, and which is configured and arranged to provide an output indicative of an operating condition of said apparatus.

6. The apparatus of claim 1, further comprising a pressure sensor disposed within said first fluid pathway and which generates an output signal corresponding to a gas pressure within said first fluid pathway.

7. The apparatus of claim 1, further comprising at least two one-way fluid valves or check valves, including a first one-way valve that allows said breathing gas to be discharged by the second fluid pump but prevent the return of said breathing gas from returning to the second fluid pump after it has been discharged, and a second one-way valve that allows said expiratory output volume to leave said second fluid pump but prevents said expiratory output volume from returning to the second fluid pump after it has been discharged.

8. The apparatus of claim 1, further comprising a bio filter in-line with said first fluid pathway and which is configured and arranged to capture bio materials within said first fluid pathway.

9. The apparatus of claim 1, wherein said drive shaft is configured and arranged to directly and mechanically force a pumping of said two fluid pumps in phase with one another.

10. The apparatus of claim 1, wherein said two pumps are configured and arranged to operate in phase with one another such that both the first and second pumps are in a first (expanded) state during said first phase of operation, and both the first and second pumps are in a second (compressed) state during said second phase of operation.
